(19) United States
(12) Patent Application Publication
(10) Pub. No.: US 2018/0289355 A1
(43) Pub. Date: Oct. 11, 2018

(54) ULTRASOUND STANDOFF DEVICE

(71) Applicant: The Board of Regents of the University of Texas System, Austin, TX (US)

(72) Inventor: Luiz MARACAJA, San Antonio, TX (US)

(73) Assignee: THE BOARD OF REGENTS OF THE UNIVERSITY OF TEXAS SYSTEM, Austin, TX (US)

(21) Appl. No.: 15/765,965

(22) PCT Filed: Oct. 5, 2016
(86) PCT No.: PCT/US2016/055463

§ 371 Date: Apr. 4, 2018

Related U.S. Application Data
(60) Provisional application No. 62/237,377, filed on Oct. 5, 2015.
(60) Provisional application No. 62/338,903, filed on May 19, 2016.

(51) Int. Cl. A61B 8/00; A61B 8/08

(52) U.S. Cl. CPC A61B 8/4411; A61B 8/0841; A61B 8/46; A61B 8/4444; A61B 8/4281

(57) ABSTRACT

Certain embodiments are directed to an ultrasound standoff device having a mounted chamber providing for ultrasound guidance for placing intervention devices, as well as methods for using the device.

This Application claims priority to U.S. Provisional Patent Applications 62/237,377 filed Oct. 5, 2015 and 62/338,903 filed May 19, 2016, each of which is incorporated herein by reference in its entirety.

### BACKGROUND

Ultrasound guidance is an essential part of current clinical practice. Multiple procedures such as vascular access (arterial or venous), nerve blocks, various types of biopsies, and various types of cavity drainage use ultrasound for guidance during the procedure. In a typical ultrasound guided procedure, a doctor will place an ultrasound transducer on a patient's skin. The transducer converts sound waves into electrical signals which are used to form a real time two-dimensional ultrasound image of a portion of the patient's body. This ultrasound image may be used to assist a health professional with locating a point where an invasive medical device, e.g., a needle, is inserted. After locating the correct insertion point, the health professional may then begin the medical procedure, such as insertion of a catheter, administration of a local anesthetic, or removal of tissue as in a biopsy.

Before beginning a procedure, it is necessary to cover the ultrasound device to assure that sterility is maintained during the procedure. Typically, a sterile sleeve made of a flexible, sterilizable material is draped over the device to form a sterility barrier. A sterility barrier is intended to refer to a seal, bond, covering, etc. that is effective in preventing micro-organisms or other contaminates from migrating from within the sleeve to the exterior, sterile environment. An acoustic coupling gel is placed in the sleeve or on the transducer before placing the transducer in the sleeve to ensure consistent contact between the sound transmitting or receiving end or head of the transducer and the sleeve. This contact is necessary to ensure there are no artifacts in the ultrasound image due to the presence of air pockets between the head and sleeve. The coupling gel may be applied to the sleeve at the time of the procedure or the gel may be pre-applied. The end of the transducer opposite the head of the transducer can be sealed once the transducer is in place.

Brackets are sometimes provided with an ultrasound device for purposes of mounting a needle guide. The needle guide is used to facilitate a longitudinal or transverse type ultrasound guided needle insertion procedure. In the “transverse” type, the guide is arranged so that the needle is inserted into the patient along a plane transverse to the ultrasound image plane. In the “longitudinal” type, the needle is inserted into the patient in a plane parallel to the ultrasound image plane.

There remains a need for additional devices for proper insertion of invasive devices.

### SUMMARY

The ultrasound standoff device having a mounted chamber provides for ultrasound guidance and flexibility as to approach and/or angle for placing intervention devices. The standoff chamber allows alignment between the needles or similar instruments with the desired target structure before skin entrance. In certain aspects the standoff device can provide for a marking device to be used during visualization to mark an area of interest on a surface that is in contact with the ultrasound probe. In a further aspect the standoff device can provide for positioning of a therapeutic device, such as a radio or ultrasonic ablative device. The standoff device can be used for real time ultrasound guided epiaortic cannulation or true lumen cannulation in cardiac operations, reducing the incidence of stroke, as well as a multitude of other procedures to increase precision and safety.

Certain embodiments of the invention are directed to an ultrasound standoff device for an ultrasound probe. The standoff device comprises a connector that is coupled to a chamber or cell formed by a support structure, the chamber or cell comprising an ultrasound or conducting medium. The medium contains at least one preformed pathway that can be form by slicing through a portion of the medium or molding an access channel or slit in the medium prior to insertion in the standoff device. In certain aspect a preformed path is horizontal to the transducer, longitudinal to the transducer or has both a horizontal and longitudinal preform pathways. The intervention device is either blunt or has a cover to blunt the interventional device while passing through the preformed pathway. A preformed pathway can reduce the probability of a medium generated embolism during the procedure. In certain aspects the ultrasound connector can be made of hard plastic or polymer. The connector is configured to receive and lock in place an ultrasound transducer. In certain aspects the connector is also configured to allow removal of the transducer after use, thus the connector is configured to be removeably connected to an ultrasound transducer. The support structure forms one or more openings or channels to allow passage of a medical intervention device through the ultrasound medium. In certain aspects the device is configured to include a port for a marking device or a therapeutic device. In further aspects the standoff device has an integrated marking device that can be deployed during an ultrasound procedure. The support structure connects the connector to the base of the device forming a chamber or cell that is filled with a conducting medium. The base forms an opening that provides for contact between the conducting medium and the surface of a target so that sounds waves can be transmitted to and/or received from the target. The support can comprise one or more wall or leg portions that connect the connector to the base.

The wall or leg portions form openings to allow access to and through the conducting medium so that an interventional device, e.g., a needle, a marking device, or a therapeutic device can be inserted through the conducting medium and into or onto a target under ultrasound guidance. The opening can be any shape and can have a height from the lower edge of the connector to the top edge of the base or any distance there between. In certain aspects the opening(s) are straight or curved and can have access points to allow for the insertion or removal of an instrument (e.g., an open slot configuration). In certain aspects the base is able to flex and contour to the surface contours of a target.

The circumference or width of the base can be larger than or equal to the circumference or width of the connector (forming a pyramidal or rectangular cube shape). In certain aspects the interior angle formed between the support and the connector is obtuse and the interior angle formed between the support and the base is acute. In other aspects the angle can be approximately right angles. In certain aspects the support-connector angle is between 45 to 160 degrees. In a further aspect the support-connector angle is between 110 and 155 degrees. The support-base angle can be between 85 to 20 degrees. In a further aspect the support-base angle is 65 to 25 degrees. The chamber or cell formed by the device is generally a cone or pyramid shape, having a width that is larger at the base than at the top. The supports or walls of the chamber need not be straight and can be curved or partially curved.

The openings at the base and in the support can be closed or covered by a film that conducts sounds waves and maintains the conducting medium in the chamber. In other aspects the consistency of the conducting medium may be such that a film is not needed and the conducting medium is formed or cast into place within the chamber or cell, as such contacts the target surface directly or indirectly through the surface of a sleeve containing the standoff device.

In another aspect, a method for inserting a needle into a body includes the steps of providing an ultrasound standoff device as described herein, positioning a device to be inserted into a target, inserting the device to be inserted into an imaged location.

The standoff device allows visualization of a device to be inserted (e.g., a needle) for positioning the device to be inserted inside the ultrasound medium before insertion towards a target structure. In certain embodiments a standoff device can be a dedicated epiaortic standoff device designed for ultrasound survey of the ascending aorta, facilitating precise real-time avoidance of atheromatous plaque disruption during placement of aortic cannulation wire for cardiopulmonary bypass (CPB) and thereby reducing risk of devastating embolic stroke.

Other embodiments are directed to arterial access and treatment of aortic dissection. The standoff device allows visualization of a device to be inserted (e.g., a needle) for positioning the device to be inserted inside the ultrasound medium before insertion towards a target structure during arterial cannulation during the treatment of an aortic dissection. In certain embodiments a standoff device can be a dedicated aortic dissection standoff device designed for ultrasound survey of an aorta dissection, facilitating precise real-time cannulation of the true lumen of the aorta and avoiding cannulation of the false lumen.

In other embodiments the standoff device can be incorporated into a sterile cover or kit configured to be connected to an ultrasound probe.

Certain embodiments are directed to a device configured to detect or visualize fluid flow. In this embodiment the device is configured to have a hook or slot into which a vessel or other tubular object can be placed and/or held. In certain aspects the hook or slot is integrated into the standoff. In other aspects the hook or slot is provided as an attachment that can be removeably positioned on the base of a standoff device. Aspects where the hook or slot is integrated into the device the device can taper from the connection to the base. In operation a vessel or tubular target is placed in the hook or slot where the ultrasound probe is positioned to monitor flow or other characteristics of the target positioned in the hook or slot of the device.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates a side view of one embodiment of an ultrasound standoff device.

FIG. 2 Illustrates a bottom up view of one embodiment of an ultrasound standoff device.

FIG. 3 Illustrates a head on side view from of one embodiment of an ultrasound standoff device.

FIG. 4 Illustrates a back on side view of one embodiment of an ultrasound standoff device.

FIG. 5 Illustrates a top down view of one embodiment of an ultrasound standoff device.

FIG. 6 Illustrates a perspective view of one embodiment of an ultrasound standoff device.

FIG. 7 Illustrates one embodiment of an ultrasound standoff device engaging the surface of an imaging target.

FIG. 8 Illustrates an exploded view of another embodiment of an ultrasound standoff device incorporated into a sterile kit device.

FIG. 9 Illustrates an assembled view of the embodiment introduced in FIG. 8.

FIG. 10A-10D. Illustrates a second embodiment of a standoff device for ultrasonography. (A) A top perspective view of a standoff device having various access points and instrument guides or clips. (B) A bottom perspective view of a standoff device having various access points and instrument guides or clips. (C) A front side view of a standoff device having various access points and instrument guides or clips. (D) A left side view of a standoff device having various access points and instrument guides or clips.

FIG. 11A-11C. Illustrates a “Flowsure” embodiment of the standoff device. (A) A bottom perspective view of a Flowsure standoff device. (B) A back side view of a Flowsure standoff device. (C) A top perspective view of a Flowsure standoff device.

FIG. 12A-12D. Illustrates a marking device embodiment of the standoff device. (A) A top perspective view of a standoff device incorporating a marking port. (B) A bottom perspective view of a standoff device incorporating a marking port. (C) A side view of a standoff device incorporating a marking port. (D) A top view of a standoff device incorporating a marking port.

FIG. 13. Exploded view of a marking device to be used in conjunction with the standoff device with a marking port.

### DESCRIPTION

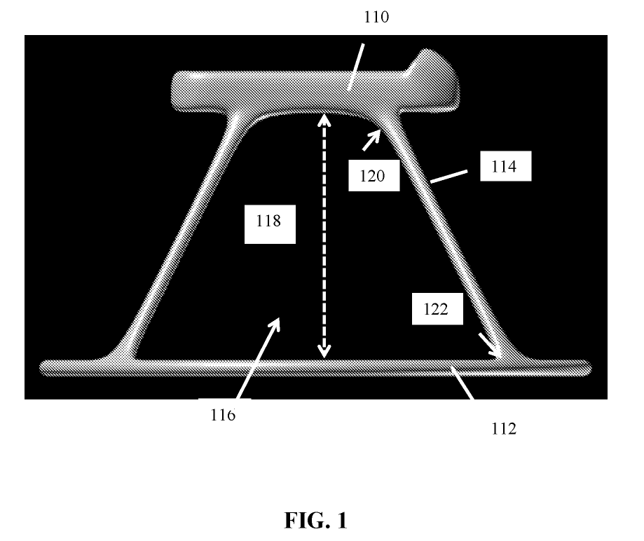

FIG. 1 to FIG. 6 show various views of one embodiment of an ultrasound standoff device. FIG. 1 is side view relative to connector 110. Connector 110 is coupled to base 112 by support(s) 114 forming a chamber or cell with in support(s) 114. In certain aspects the supports can be configured as sidewalls. Support(s) 114 form access point 116 that provide for the insertion of an intervention devices such as needles and the like. Support(s) 114 are shown in FIG. 1 as a plurality of legs extending at a predetermined angle from the connector (forming support-connector angle 120) to the base (forming support-base angle 122). In other embodiments the support can be of a width sufficient to be characterized as a wall having an access opening formed in the wall or between wall components. Thus, the “legs” of the support need not be of any particular width in that the legs may be of width sufficient to form a wall of the chamber or cell. Support(s) 114 maintain the connector at a distance 118 from the base and the target. Connector 110 comprises a locking or snapping mechanism that engages an ultrasound transducer and locks the standoff device onto the transducer for operational purposes. In certain aspects the transducer can be removed from the standoff device after use.

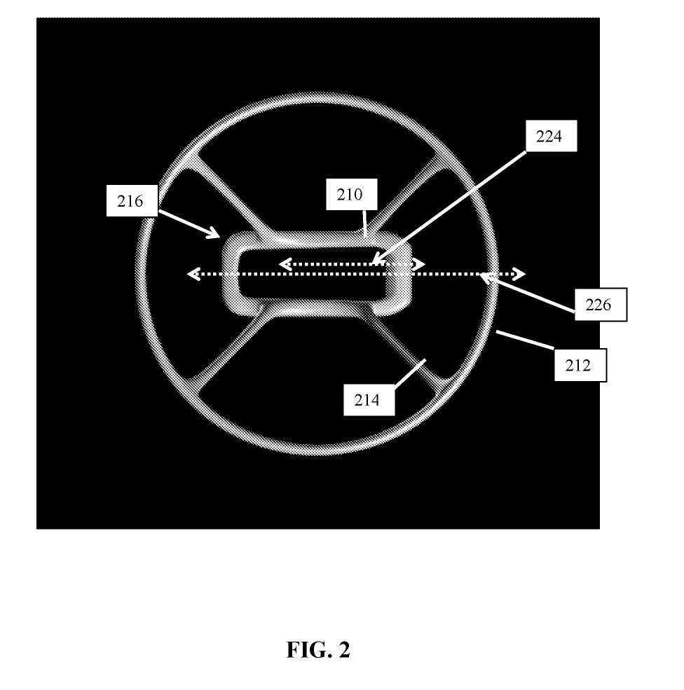

FIG. 2 is bottom up view of the embodiment of the standoff device introduced in FIG. 1. Connector 210 is connected to base 212 by supports 214 forming a chamber or cell. The chamber or cell can be filled with a conducting medium that is permissive for sound wave propagation through the chamber or cell. Sound waves can travel from the transducer engaged at connector 210 to base 212 and/or from base 212 to the transducer. Connector 210 has an opening with a length 224 that is configured to provide for contact between a transducer and conducing medium filling the chamber or cell. The base of FIG. 2 has a length 226. Base 212 can have a regular or irregular shape that can be circular, elliptical, square, rectangular, rhomboid, or any other polygonal shape or curve. In certain aspects base 212 can flex to conform to the surface contours of a target. In other embodiments the conducting medium in the chamber or cell extends beyond base 212 and is malleable enough to conform with the surface contours of a target.

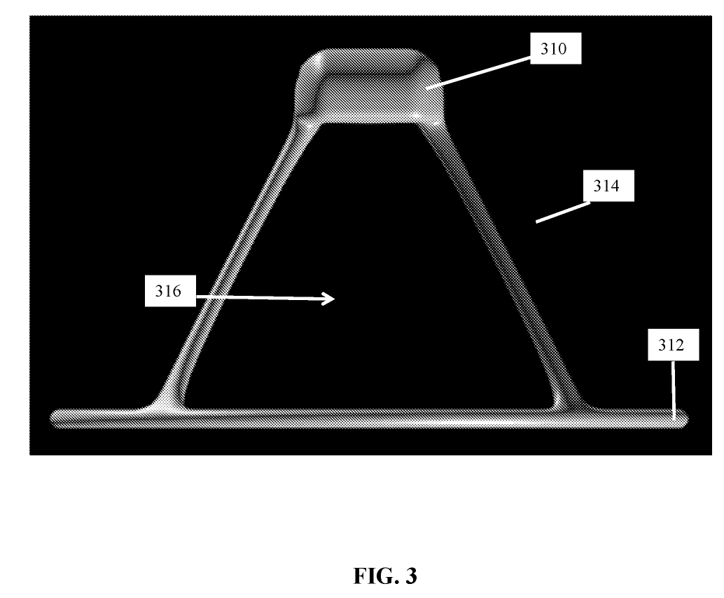

FIG. 3 shows a side view of the embodiment illustrated in FIG. 1. Connector 310 is connected to base 312 by supports 314 forming a chamber or cell. Connector 310 is configured to receive and secure an ultrasound probe. Also shown is access opening 316 between supports 314 that provides access to the chamber or cell of the device.

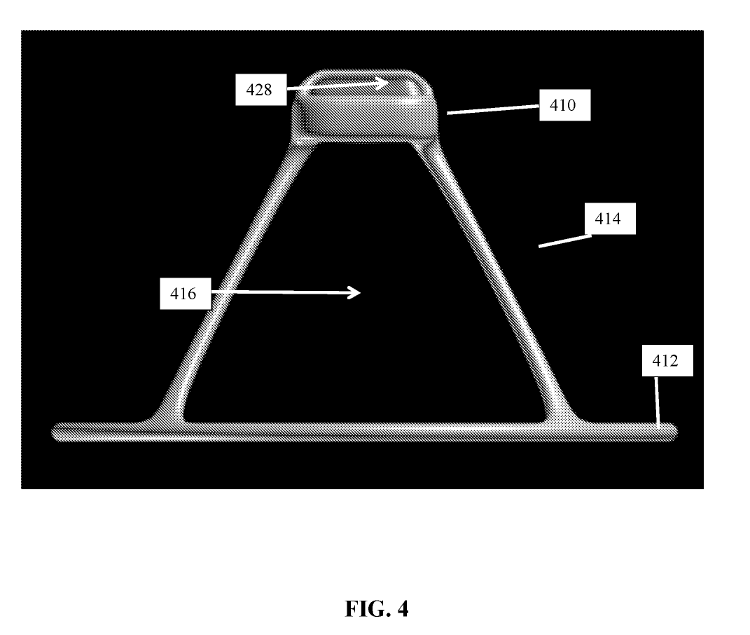

FIG. 4 shows a back on view (180 degrees from head on view) of the embodiment of the device depicted in FIG. 1. Connector 410 is connected to base 412 by supports 414 forming a chamber or cell. Connector 410 is configured to receive and secure an ultrasound probe. Receiving lip 428 is configured to receive an secure an ultrasound probe. Also shown is access opening 416 between supports 414 that provides access to the chamber or cell of the device.

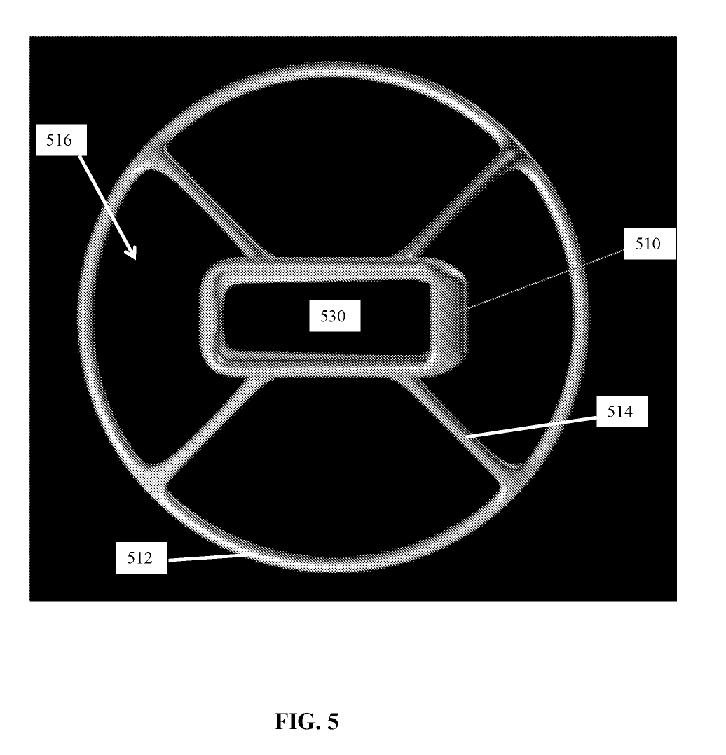

FIG. 5 shows a top down view of the embodiment of the device depicted in FIG. 1. Connector 510 is connected to base 512 by supports 514 forming a chamber or cell. Access points 516 are formed by supports 514. Also shown is transducer window 530 that allows for the ultrasound probe to contact the conducting medium in the chamber or cell.

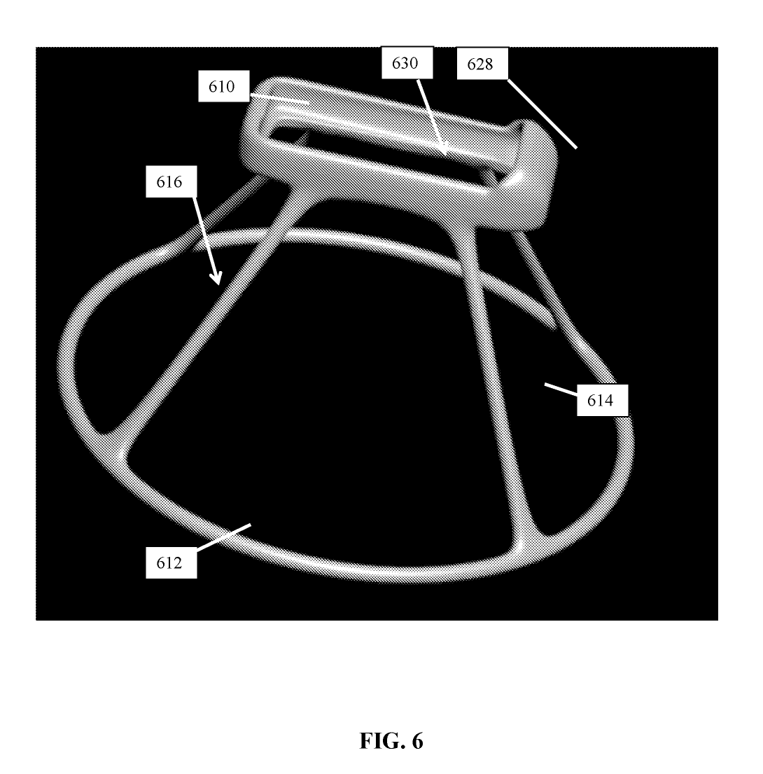

FIG. 6 shows a perspective view of the embodiment of the device depicted in FIG. 1. Connector 610 is connected to base 612 by supports 614 forming a chamber or cell. Access points 616 are formed by supports 614. Also shown is receiving lip portion 628 and transducer window 630 of connector 610.

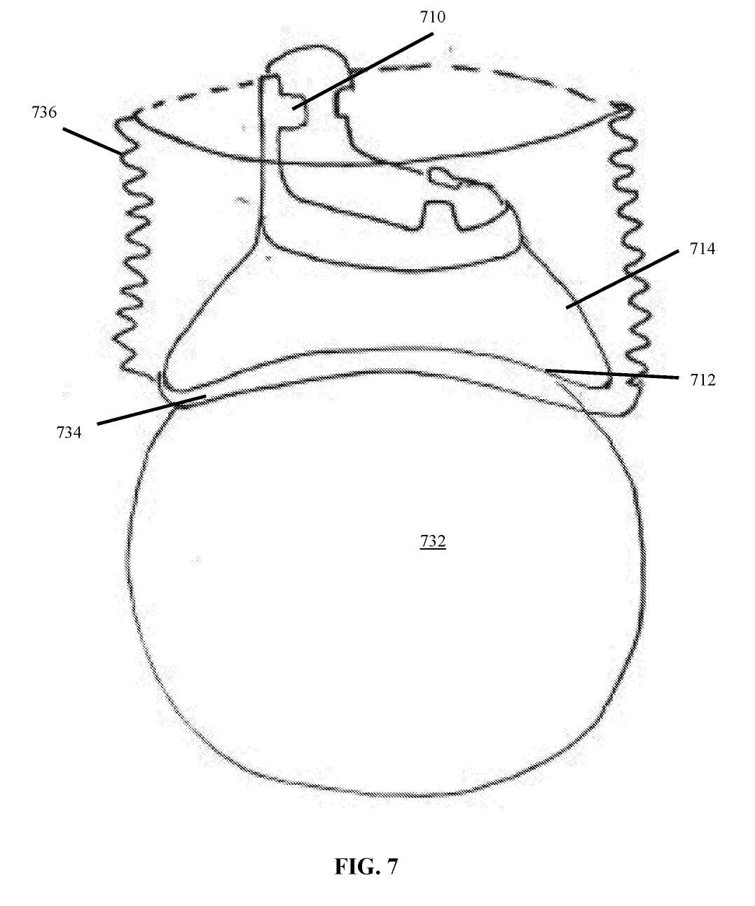

FIG. 7 shows a view of the device depicted in FIG. 1 in contact with a target. Connector 710 is connected to base 712 by supports 714 forming a chamber or cell. Access points are not illustrated in this particular drawing. Base 712 can flex to conform to the surface of target 732 or the conducting medium 734 can extend beyond the chamber or the base and conform to the contours of the target surface. Also depicted in FIG. 7 is sterile sleeve 736.

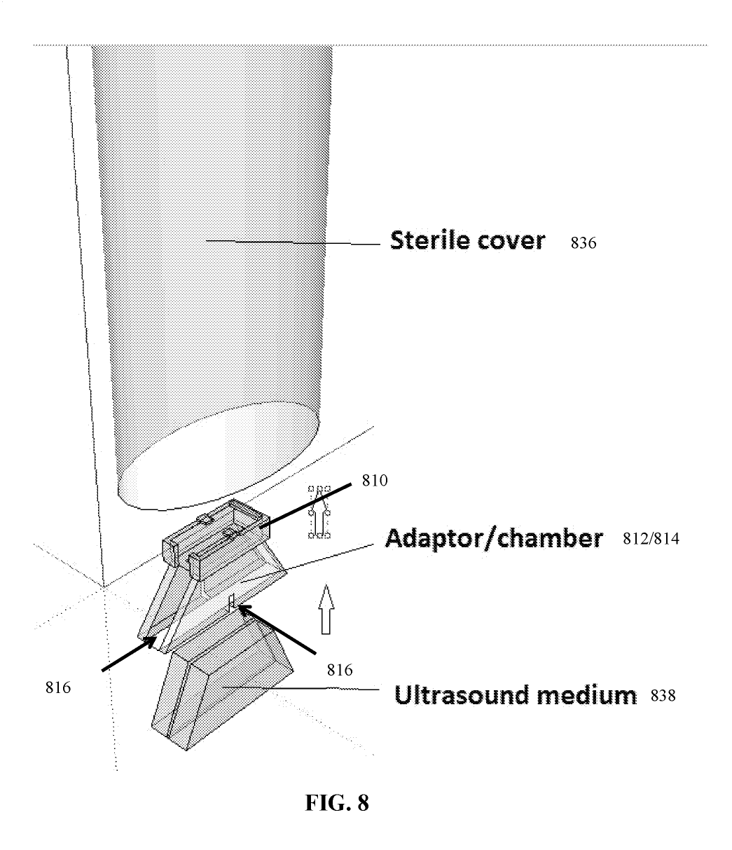

FIG. 8 illustrates an exploded view of one embodiment of the ultrasound standoff (810, 812, 814) with sterile sleeve or cover 836. Sterile cover 836 in the embodiment illustrated is attached to connector 810 forming a sterile seal. Connector 810 is connected to the chamber formed by base 812 and supports 814. Support 816 forms access opening 816. Ultrasound or conducting medium 838 having at least one preformed pathway fills the chamber formed by connector 810, base 812, and support 814.

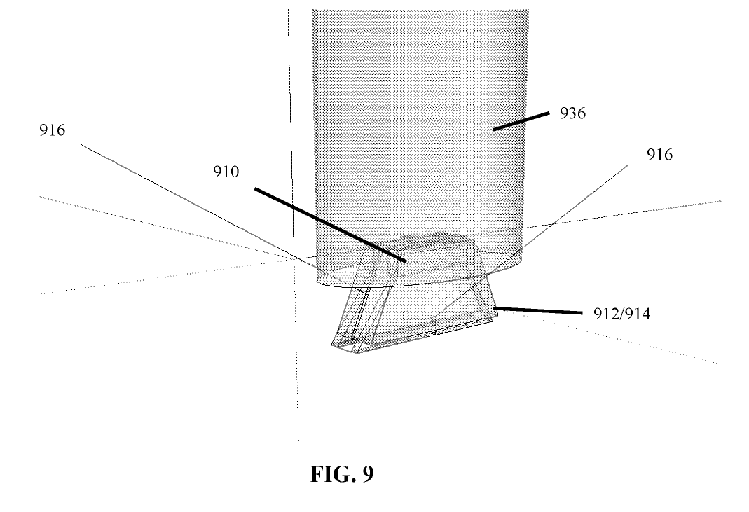

FIG. 9 illustrates an assembled embodiment of the ultrasound standoff (910, 912, 914) with sterile sleeve or cover 936. Sterile cover 936 in the embodiment illustrated is attached to connector 910 forming a sterile seal. Connector 910 is connected to the chamber formed by base 912 and supports 914. Support 914 forms access openings 916. Ultrasound or conducting medium 938 fills the chamber formed by connector 910, base 912, and support 914 and form a chamber that is filled conducting medium.

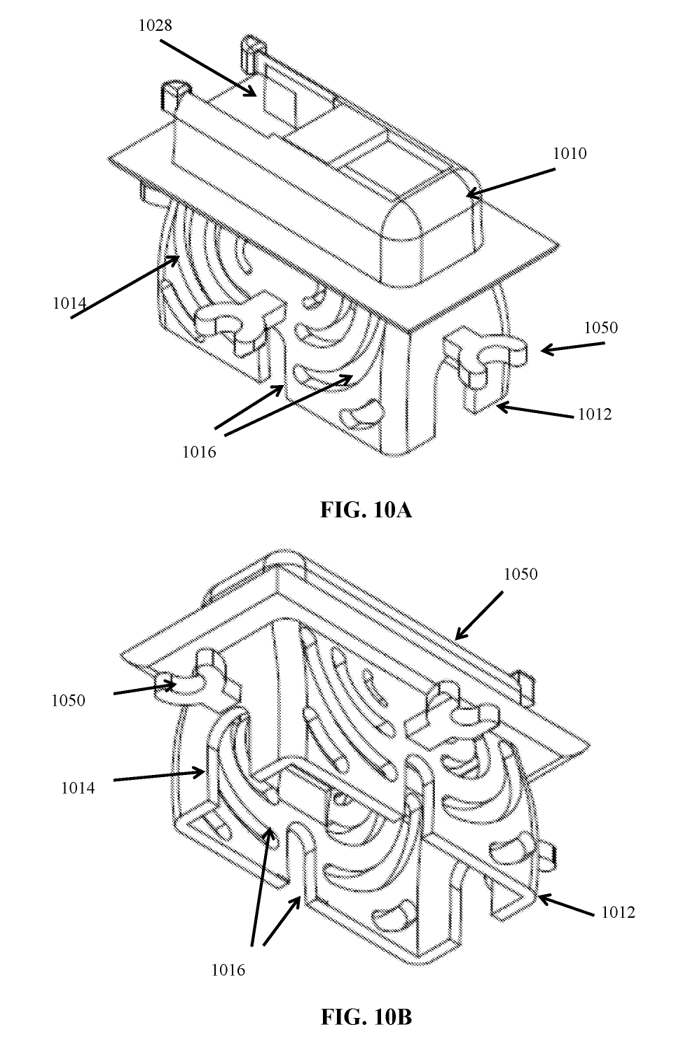

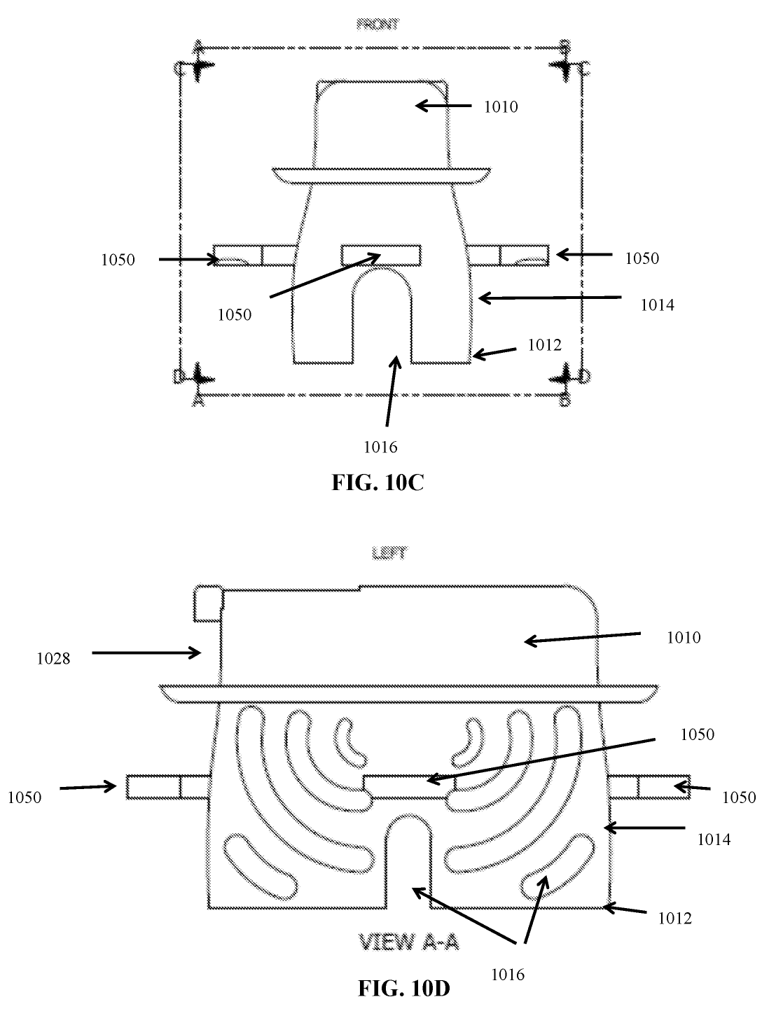

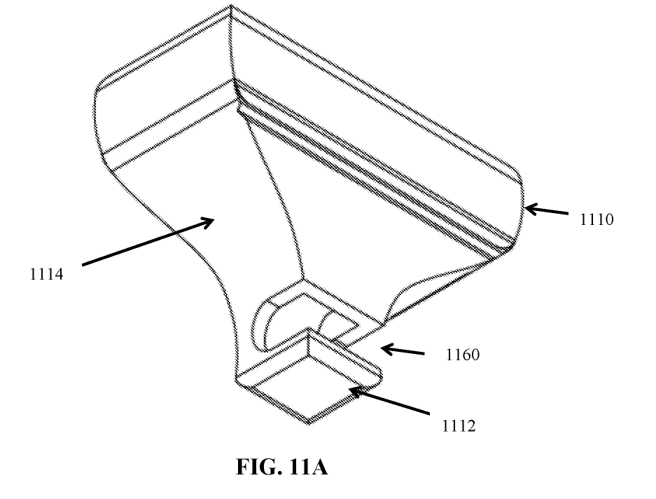

FIG. 10A illustrates top perspective view another embodiment of a standoff device. In this embodiment the chamber or cell is approximately a rectangular prism having a base 1012 and sidewalls 1014. Access openings 1016 are provided in the sidewall 1014. Attachment, guide or clip 1050 can be included as part of the exterior of sidewall 1014. Connector 1010 is configured to receive a sonogram probe and couple the probe to a coupling medium filling the chamber or cell formed by sidewalls 1014. Base 1012 is configured to allow coupling of a target to a probe through a coupling medium. A probe can be attached through probe access opening 1028.

FIG. 10B illustrates bottom perspective view of a standoff device. In this embodiment the chamber or cell is approximately a rectangular prism having a base 1012 and sidewalls 1014. Access openings 1016 are provided in the sidewall 1014. Attachment, guide or clip 1050 can be included as part of the exterior of sidewall 1014. Connector 1010 is configured to receive a sonogram probe and couple the probe to a coupling medium filling the chamber or cell formed by sidewalls 1014. Base 1012 is configured to allow coupling of a target to a probe through a coupling medium.

FIG. 10C illustrates side view of a standoff device along the long axis of the device. In this embodiment the chamber or cell is approximately a rectangular prism having a base 1012 and sidewalls 1014. Access openings 1016 are provided in the sidewall 1014. Attachment, guide or clip 1050 can be included as part of the exterior of sidewall 1014. Connector 1010 is configured to receive a sonogram probe and couple the probe to a coupling medium filling the chamber or cell formed by sidewalls 1014.

FIG. 10D illustrates side view of a standoff device along the short axis of the device. In this embodiment the chamber or cell is approximately a rectangular prism having a base 1012 and sidewalls 1014. Access openings 1016 are provided in the sidewall 1014. Attachment, guide or clip 1050 can be included as part of the exterior of sidewall 1014. Connector 1010 is configured to receive a sonogram probe and couple the probe to a coupling medium filling the chamber or cell formed by sidewalls 1014.

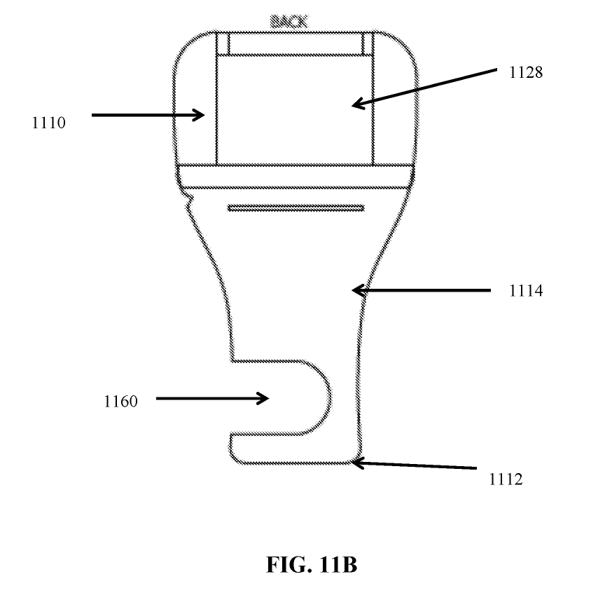

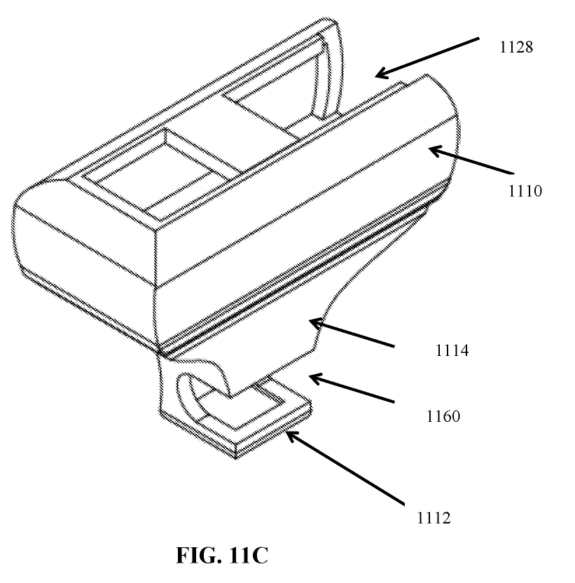

FIG. 11 illustrates one embodiment of the Flowsure adaptation of the standoff device. FIG. 11A shows a bottom perspective illustrating the hook, slot, or notch used for analyzing flow in a target position in slot 1160. Connector 1110 is shown at the top with sidewalls 1114 adapted to taper the base 1112 which, with the sidewall forms slot 1160. FIG. 11B is a side view along the long axis of the standoff device. Slot 1160 is configured to receive a target having a lumen, such as a vessel, for analyzing flow in the target using a sonogram probe. The probe is attached via connector 1110 that has opening 1128 for the attaching the probe and coupling the probe to a coupling medium that can be placed in the cell or chamber formed by sidewall(s) 1114. Sidewalls 1114 taper to base 1112 which forms slot 1160. FIG. 11C is a top perspective view of the Flowsure standoff device. Slot 1160 is configured to receive a target having a lumen, such as a vessel, for analyzing flow in the target using a sonogram probe. The probe is attached via connector 1110 that has opening 1128 for the attaching the probe and coupling the probe to a coupling medium that can be placed in the cell or chamber formed by sidewall(s) 1114. Sidewalls 1114 taper to base 1112 which forms slot 1160.

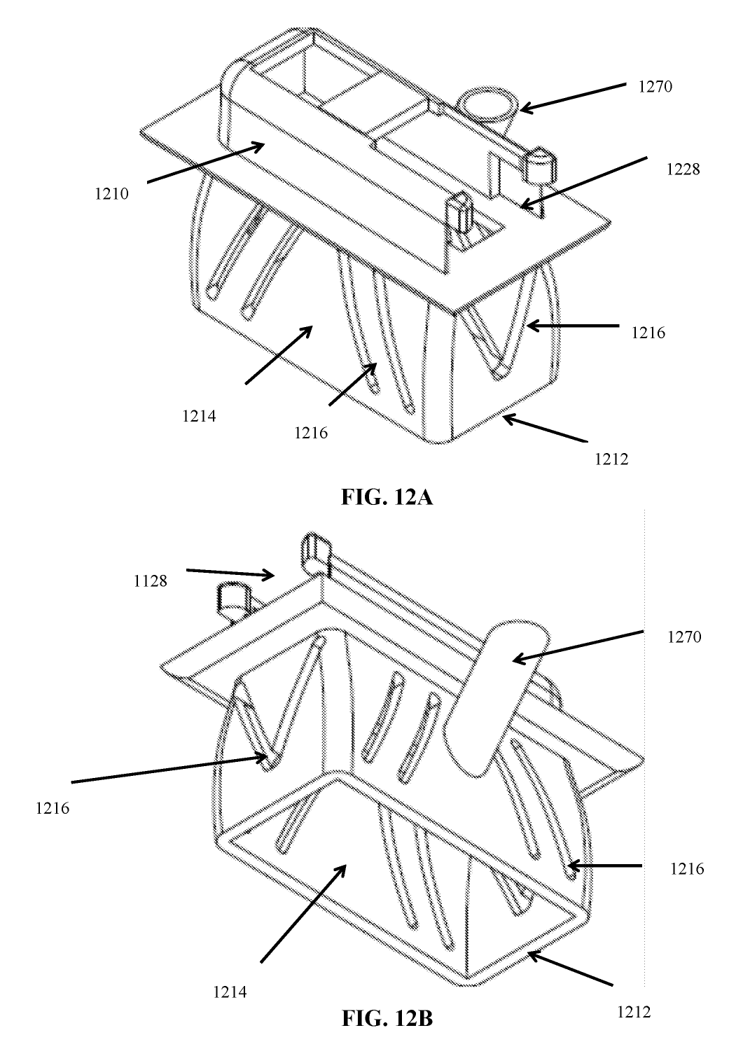

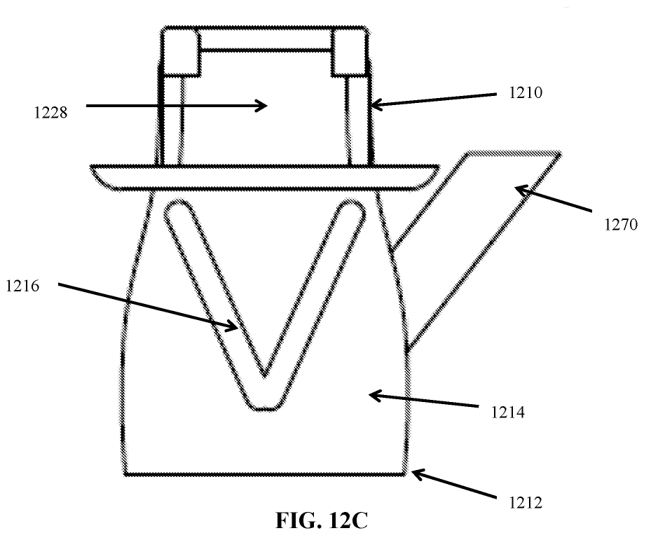

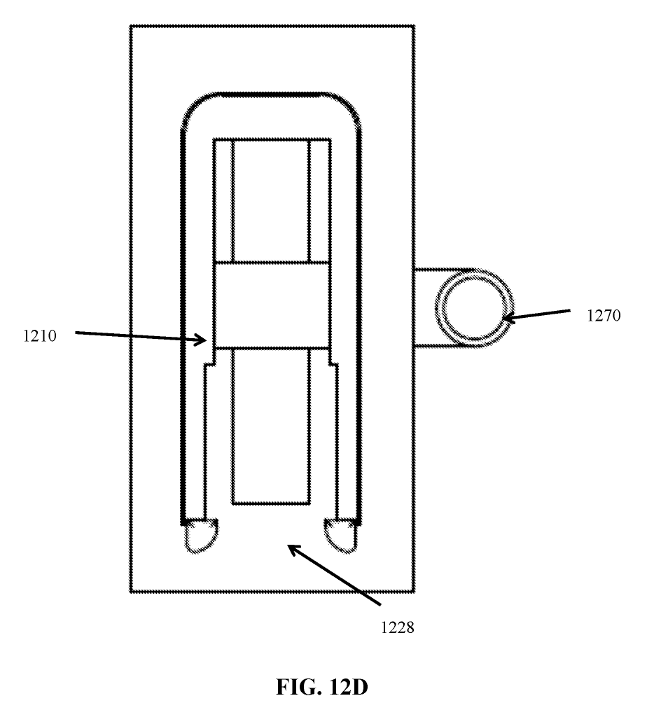

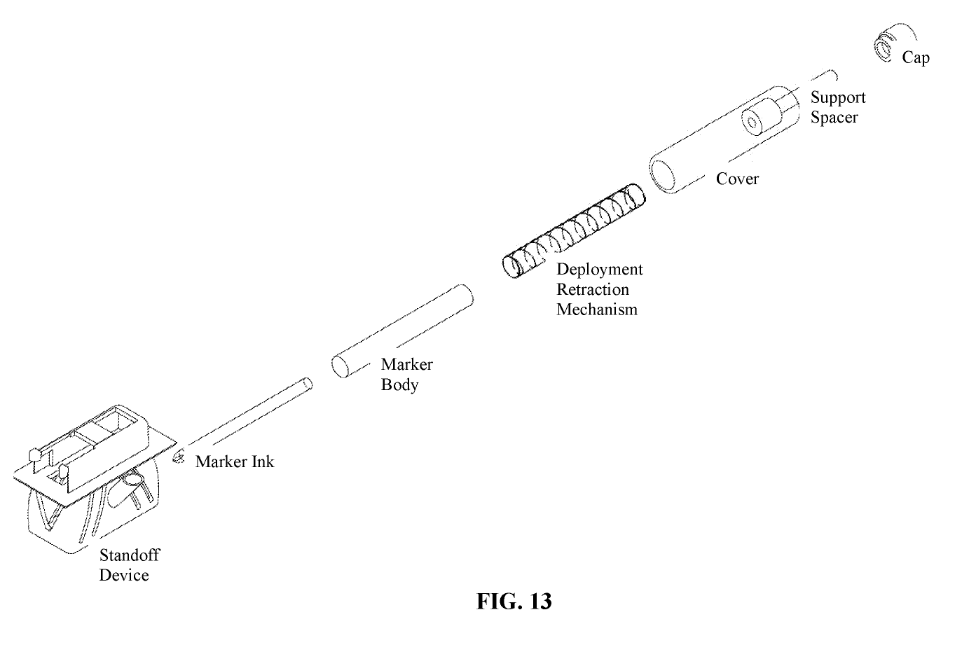

FIG. 12 illustrates a standoff device having a marking port incorporated into the device. FIG. 12A shows a top perspective view of such a device. Marking port 1270 is included to allow the user to apply a mark to the surface of a target. Marking port 1270 is coupled to the chamber or cell of a standoff device. In this particular embodiment the chamber is approximately a rectangular prism having a base 1212 and sidewalls 1214. Access openings 1216 are provided in the sidewall 1214. Marking port 1270 can be included as part of the exterior of sidewall 1214, forming a lumen through which a marking device can be inserted. Connector 1210 is configured to receive a sonogram probe and couple the probe to a coupling medium filling the chamber or cell formed by sidewalls 1214. FIG. 12B shows a bottom perspective view of such a device. Marking port 1270 is coupled to the chamber or cell of a standoff device. In this particular embodiment the chamber is approximately a rectangular prism having a base 1212 and sidewalls 1214. Access openings 1216 are provided in the sidewall 1214. Marking port 1270 can be included as part of the exterior of sidewall 1214, forming a lumen through which a marking device can be inserted. Connector opening 1228 is configured to receive a sonogram probe and couple the probe to a coupling medium filling the chamber or cell formed by sidewalls 1214. FIG. 12C shows a side view of such a device along the long axis. Marking port 1270 is coupled to the chamber or cell of a standoff device. In this particular embodiment the chamber is approximately a rectangular prism having a base 1212 and sidewalls 1214. Access openings 1216 are provided in the sidewall 1214. Marking port 1270 can be included as part of the exterior of sidewall 1214, forming a lumen through which a marking device can be inserted. Connector 1210 is configured to have opening 1228 to receive a sonogram probe and couple the probe to a coupling medium filling the chamber or cell formed by sidewalls 1214. FIG. 12D shows a top view of such a device. Marking port 1270 is included to allow the user to apply a mark to the surface of a target. Marking port 1270 is coupled to the chamber or cell of a standoff device. Connector 1210 is configured to receive a sonogram probe and couple the probe to a coupling medium filling the chamber or cell formed by sidewalls. During operation a marking device can be deployed through a path in the coupling to the surface of a target being imaged by sonography. A mark can be made on the surface of a target. FIG. 13 shows an exploded view of a marking device that can be coupled to a standoff device through a marking port. The marking device can comprise marker ink or a marking portion that is housed in a marker body. Inside the marker body the marking portion is operatively coupled to a deployment/retraction mechanism. The deployment mechanism is coupled to a support spacer and a cap. The cap can be depressed to deploy or retract the marking portion to or from the target surface.

The connector may include an elastic edge or recess that snaps into a mating recess or ridge on an ultrasound probe. The engagement may be such that an audible confirmation is provided when proper engagement is achieved, e.g., by a “clicking” sound. This confirmation may be desirable for the purpose of giving assurance that the ultrasound probe is aligned with the ultrasound window of the connector as well as providing for appropriate positioning of the probe and access opening in the standoff. In operation the standoff used in conjunction with an ultrasound imaging system produces a visual image of a target and assist in aligning an intervention device or other instruments with internal structures and the appropriate path to those internal structures.

Pre-sterilized medical procedure kits are known and used for various medical procedures. Such sterilized procedure kits are provided with a plurality of components used in connection with a particular surgical procedure. Certain embodiments are directed to sterilized surgical kits to maintain a sterial environment or reduce the risk for infection during a procedure. The kits can include a sterile sleeve or cover for the ultrasound components used in ultrasound guided procedures. In certain aspects the sleeve is configured with an opening for an ultrasound transducer or probe. The opening can also be configured to provide access for insertion of a needle or other device while imaging with ultrasound. The opening can be configured with a sterile transducer cover attached, thus the sleeve or cover will have an open end and a closed end. In certain embodiments the open is configured to be sealed or closed to some extend to aide in maintaining sterility or to minimize contamination of the immediate environment.

A kit can include a sleeve or cover with attached standoff device contained with a sterile, sealed compartment that is configured to be accessed when need for the kit arises. When accessed an apperture is exposed to allow insertion of the ultrasound transducer into ultrasound cover and connected to the standoff device prior to imaging.

Prior to using the kit and/or standoff device auxilary equipment or devices will need to be assembled and/or prepared. In certain aspects an ultrasound device with its tranducer will need to be acquired and prepped for use. In certain aspect an ultrsound conducting material is positioned inside and outside of the sleeve or cover in order to enhance ultrasound transmission to the transducer. Any materials that will be in contact with the patient can be provided in sterial compartments or packaging that can be opened just prior to use in order to maintain sterility or reduce contamination.

The standoff chamber is composed of an outside frame made of hard or semi-rigid material such as plastic that can be adapted to an ultrasound probe. This chamber will be the support structure for the internal ultrasound compatible medium such as fluid, gel, gelatinous materials of different densities or even solids. The ultrasound chamber frame can have different forms, shapes and sizes depending on the ultrasound probe it will be attached to and the purpose or procedure to be performed. Once attached to the ultrasound probe, it will act as an extension of the transducer.

In order to be used in surgeries or interventional procedures, the standoff chamber components can be sterilized by different methods such ethylene oxide or flash steam sterilization. In the opposite side of the frame that adapts to the ultrasound transducer, a sterile sleeve or cover for the ultrasound probe cable can be attached and sealed to the standoff which will provide a sterile barrier to the probe and respective cable.

The ultrasound compatible medium with gelatinous consistency such as plastisol, alumisol, silicone or biocompatible gelatin can be used to couple the target with an ultrasound probe through the standoff device. An important discovery and aspect of the embodied ultrasound medium is that very thin slices, cuts, paths or punctures can be made in the medium without significant interference with the ultrasound transmission and image.

This happens because the external frame maintains the two sliced parts coupled thereby eliminating the formation of air pockets. This feature allows placement of different structures such as needles, marking pens, magnetic or electromagnetic metals, catheters, surgical and interventional instruments, veins, arteries, grafts, or any anatomic structure in which ultrasound imaging is desired.

When used with needles inside the chamber, the standoff device allows the physician performing the ultrasound guided procedure to visualize the needle and perform adjustments in insertion angle based on the imaging underneath the chamber. The cuts in the ultrasound material are made longitudinally and transversely thereby crossing the midline of the ultrasound array.

When used with a marking pen, the standoff device, aka. “Sonopen” (for example see FIG. 12), allows marking the surface of desired region based on the imaged structure underneath the chamber. This feature has potential clinical applications such as epiaortic scanning in cardiac surgery, demarcation of tumor resection margins in different organs, venous mapping for vein harvest or resection of varicose veins, or for planning surgical incision in different operations. A retractable pen mechanism is also part of the embodiment which allows easy, precise and multiple site marking.

When used to measure blood flow inside vessels the standoff device, aka “Flowsure” (for an example see FIG. 11), allows the longitudinal alignment of the vessel with the ultrasound array. This feature allows two and three dimensional ultrasound imaging, color flow doppler, pulsated wave Doppler and continuous Doppler interrogation of flow velocities of the desired region of the vessel. This device offer many possible clinical applications such as measurement of flow velocities, resistance indices and in procedures with arterial anastomosis such as vascular procedures, liver transplants, coronary bypass grafts, and any micro-anastomosis. Also, the ultrasound imaging allows visualization of thrombus or stenosis inside the vessel lumen. Furthermore, this device can also be used as an adaptor for the evaluation of imaging and flow in tubes of different blood circuits such as extracorporeal oxygenation membrane, ventricular assist devices, cardiopulmonary bypass systems, perfusion systems for ex-vivo organ preservation for transplant, preservation system for cell cultures and tissue engineering.

When used with magnetic or electromagnetic stylet in place of a marking device, certain embodiments, e.g., the Sonomag device, allows the application of magnetic field in a specific location based on the imaging underneath the chamber. This same device could also be used for electrocautery, radiofrequency, or radiation therapy. The ultrasound can be used for finding guidewires and the stylet or needle with magnetic properties can be used for retrieve metal intravascular wires.

1. An ultrasound standoff device for guiding medical interventions comprising:
a base connected to an ultrasound connector by a support structure positioning the ultrasound connector a predetermined distance above the base;
the support structure forming (i) a cavity that is filled with an ultrasound conducting media, the media comprising at least one preformed pathway for passing an intervention device through the media, and (ii) access pathways configured to allow access to the at least one preformed pathway for insertion of an intervention device,
the ultrasound connector being configured to couple an ultrasound transducer to the ultrasound conducting media; and
the base forming an opening providing for coupling the surface of an ultrasound target to the ultrasound conducting media.

2. The device of claim 1, further comprising a marking device port configured to provide marking device access to the chamber, wherein a marking device can be inserted through the chamber and coupling medium to the surface of a target for the purpose of marking a position on the surface of the target.

3. The device of claim 2, wherein the marking device is a pen.

4. The device of claim 2, wherein the marking device port is formed in a sidewall of the chamber and comprises a guidewall for the marking device that protrudes from the sidewall of the chamber.

5. The device of claim 4, wherein the port formed by the guidewall is angled to direct the marking device to a predetermined location in the sonogram field.

6. An imaging system comprising an ultrasound standoff device of claim 1.

7. A method for real time ultrasound guided insertion of an intervention device during medical procedures comprising:
positioning a device of claim 1 that is operably connected to an ultrasound imaging system over a location for insertion of an intervention device;
imaging the portion of the subject to receive an intervention device; and
inserting an intervention device into the patient at an appropriate position using an appropriate path as determined by ultrasound imaging, wherein the intervention device is passed through an access opening in the device of claim 1.

8. The method of claim 7 wherein the intervention device is a needle or a cannula.

9. A method for real time ultrasound guided epiaortic cannulation in cardiac operations comprising:
positioning a device of claim 1 that is operably connected to an ultrasound imaging system over a location for insertion of an intervention device;
imaging the aortic portion to be cannulated; and
inserting an intervention device into the aorta of a patient through an access opening in the device of claim 1 based on ultrasound guidance.

10. An ultrasound standoff device for detecting fluid flow in a target having a lumen comprising:
a proximal base connected to an ultrasound connector by a support structure positioning the ultrasound connector a predetermined distance above the base;
the support structure forming (i) a cavity that is filled with an ultrasound conducting media, and (ii) a distal base having a hook or slot formed in the distal portion of the support structure, the hook or slot being configured to receive a target having a lumen; and
wherein the ultrasound connector is configured to couple an ultrasound transducer to the ultrasound conducting media.
